(19) United States
(12) Patent Application Publication
(10) Pub. No.: US 2025/0360185 A1
(43) Pub. Date: Nov. 27, 2025

(54) COMPOSITIONS COMPRISING PEDF-DERIVED SHORT PEPTIDES FOR THE TREATMENT OF DRY EYE DISEASES

(71) Applicants: Haishan JANG, Taipei (TW); BRIM Biotechnology, Inc., Taipei (TW)

(72) Inventors: Haishan JANG, Exton, PA (US); Sin-Tzu NING, Taipei (TW); Ya-Lang HUANG, Taipei (TW); Frank Wen-Chi LEE, Orlando, FL (US); Huei-Ling CHANG, Taipei (TW)

(73) Assignee: BRIM Biotechnology, Inc., Taipei (TW)

(21) Appl. No.: 18/875,114

(22) PCT Filed: Jun. 13, 2023
(86) PCT No.: PCT/US2023/025220

§ 371 Date: Dec. 13, 2024

Related U.S. Application Data
(60) Provisional application No. 63/351,801, filed on Jun. 13, 2022.

(51) Int. Cl. A61K 38/17

(52) U.S. Cl. CPC A61K 38/1709

(57) ABSTRACT

A pharmaceutical composition for use in treating dry eye disease includes a peptide having the amino-acid sequence selected from SEQ ID NO: 1-7. A method for treating dry eye disease, comprising: administering to a subject in need thereof a composition comprising a peptide having the amino-acid sequence selected from SEQ ID NO: 1-7. The dry eye disease is accompanied with cataract. The subject suffers from cataract that has been treated with surgery.

### BACKGROUND OF INVENTION

### Field of the Invention

The present invention relates to PEDF-derived short peptides (PDSP) and their uses in the treatment and/or amelioration of dry eye diseases.

### Background Art

Dry eye (i.e., Keratoconjunctivitis Sicca) is a complex disease that results in symptoms of discomfort, visual disturbance, and tear film instability, which creates potential for damage to the ocular surface. Dry eye occurs when the eye does not produce enough tears or when the tears evaporate too quickly. It is accompanied by increased osmolarity of the tear film and inflammation of the ocular surface. The severity of dry eye signs/symptoms varies widely among patients. While some patients suffer only minor irritation, others experience significant complications that lead to severe corneal damages and vision impairment.

Dry eye disease (DED) can arise from various reasons that affect the production of any of the tear composition (lipid, aqueous, and mucin) or stability of the tear film (e.g., fast evaporation of tears), resulting in tear hyperosmolarity, tear film instability, and inadequately support of the ocular surface epithelium integrity. Tear hyperosmolarity condition can potentially damage and stimulate inflammation cascade of ocular epithelial cells, leading to loss of surface epithelial cells, including the conjunctival goblet cells. Loss of goblet cells reduces mucin secretion, which results in loss of protection on surface epithelium and tear film instability, as well as the development of dry eye diseases and damages on corneal epithelial cells. The tear film instability often results in a chronic cycle of inflammation and damage to ocular surface that cause DED.

Currently, few therapeutic options are available for DED patients, including artificial tear, anti-inflammation drugs, and analgesics. Even though these treatments can alleviate some symptoms for some patients, there is still a need for better treatment and prevention means for DED.

### SUMMARY OF THE INVENTION

One aspect of the invention relates to pharmaceutical compositions for use in treating dry eye diseases (DED). A pharmaceutical composition for use intreating dry eye disease (DED) in accordance with one embodiment of the invention comprises a peptide having the amino-acid sequence selected from SEQ ID NO: 1-7. In accordance with embodiments of the invention, the subject suffers from DED and cataract. In accordance with some embodiments of the invention, the subject suffers from cataract that has been treated with surgery.

One aspect of the invention relates to methods for treating dry eye disease (DED) in a subject. A method for treating dry eye disease (DED) in accordance with one embodiment of the invention comprises administering to a subject in need thereof a composition comprising a peptide having the amino-acid sequence selected from SEQ ID NO: 1-7. In accordance with some embodiments of the invention, the subject suffers from DED and cataract. In accordance with some embodiments of the invention, the subject suffers from cataract that has been treated with surgery.

Other aspect of the invention will become apparent with the following description and the enclosed drawings.

### BRIEF DESCRIPTION OF THE DRAWINGS

FIG. 1 shows a schematic of treatment plan according to embodiments of the invention.

FIG. 2A shows treatment efficacies of BRM421 with respect to visual analog scale (VAS) dryness, VAS burning/stinging, and VAS photophobia. FIG. 2B shows treatment efficacies of BRM421 with respect to VAS dryness and VAS burning/stinging, analyzed with respect to the pre-CAE® and post-CAE® evaluations.

FIG. 3A shows treatment efficacies of BRM421 with respect to Diary dryness and Diary burning. FIG. 3B shows treatment efficacies of BRM421 with respect to Diary (ODS) dryness, ODS burning, and ODS stinging, analyzed with respect to the pre-CAE® and post-CAE® evaluations.

FIG. 4A shows treatment efficacies of BRM421 with respect to VAS burning/stinging in subjects without cataract. FIG. 4B shows treatment efficacies of BRM421 with respect to VAS burning/stinging in subjects with cataract. FIG. 4C shows treatment efficacies of BRM421 with respect to VAS burning/stinging in subjects with cataract and no surgery. FIG. 4D shows treatment efficacies of BRM421 with respect to VAS burning/stinging in subjects with cataract and surgery. FIG. 4E shows treatment efficacies of BRM421 with respect to VAS dryness in subjects without cataract. FIG. 4F shows treatment efficacies of BRM421 with respect to VAS dryness in subjects with cataract. FIG. 4G shows treatment efficacies of BRM421 with respect to VAS dryness in subjects with cataract and no surgery. FIG. 4H shows treatment efficacies of BRM421 with respect to VAS dryness in subjects with cataract and surgery.

FIG. 5A shows corneal damage repair in subjects without cataract. FIG. 5B shows significant improvements in corneal damage repair in the BRM421 group, as compared to the placebo group. FIG. 5C shows significant improvements in corneal damage repair in the BRM421 group, as compared to the placebo group, in patients with cataract but without surgery. FIG. 5D shows slight improvements in corneal damage repair in the BRM421 group, as compared to the placebo group, in patients with cataract and surgery.

### DETAILED DESCRIPTION OF THE INVENTION

Human Pigment Epithelium-derived Factor (PEDF) is a secreted protein containing 418 amino acids, with a molecular weight of about 50 kDa. PEDF is a multifunctional protein with many biological functions (see e.g., U.S. Patent Application Publication No. 2010/0047212). Different peptide regions of the PEDF are found to be responsible for different functions. For example, a 34-mer fragment (residues 44-77 of PEDF) has been identified to have anti-angiogenic activity, while a 44-mer fragment (residues 78-121 of PEDF) has been identified to have neurotrophic properties,

U.S. Patent Application Publication No. 2010/0047212 discloses that PEDF can promote self-renewal of stem cells, U.S. Pat. No. 9,051,547 and U.S. Pat. No. 9,617,311 disclose that fragments of PEDF having 20-39 amino acids in length (residues 93-121 of PEDF) can promote stem cells proliferation and wound healing. These PEDF-derived short peptides are referred to as PDSP in this description. PDSP used in this invention are listed in TABLE 1 below:

```
TABLE 1
LSVATALSALSLGAEQRTESIIHRALYYDL
ISSPDIHGT
(SEQ ID NO: 1; 39mer; 83-121)
          ALSALSLGAEQRTESIIHRALYYDL
ISSPDIHGT
(SEQ ID NO: 2; 34mer; 88-121)
                    SLGAEQRTESIIHRALYYDL
ISSPDIHGT
(SEQ ID NO: 3; 29mer; 93-121)
                    SLGAEQRTESIIHRALYYDL
ISSP
(SEQ ID NO: 4; 24mer; 93-116)
                    SLGAEQRTESIIHRALYYDL
(SEQ ID NO: 5; 20mer; 93-112)
                    SLGAE [symbol] RTES [symbol] IHRALYYDL
ITNPDIHST
(SEQ ID NO: 6; mo-29mer)
                    SLGAE [symbol] RTES [symbol] IHRALYYDL
(SEQ ID NO: 7; mo-20mer)
```

Embodiments of the invention relate to PDSP and their uses in the prevention and/or treatment of dry eye diseases (DED). In preclinical studies, all PDSP listed in TABLE 1 have been found to be effective in preventing and/or treating DED. The following specific examples will use results from clinical trial of the 29-mer (SEQ ID NO: 3, hereafter referred to as “BRM421”) to illustrate embodiments of the invention, while other PDSP listed in TABLE 1 also have similar effects. One skilled in the art would appreciate that the results of the BRM421 are for illustration and are not intended to limit the scope of the invention.

A multi-center, randomized, double masked, placebo controlled clinical study was conducted to assess the safety and efficacy of BRM421 ophthalmic solution (OS) in subjects with DED using a controlled adverse environment (CAE®) model (Ora, Inc., Andover, MA, USA). CAE® uses controlled ocular surface stress to exacerbate the signs and symptoms of DED in a safe and controllable manner.

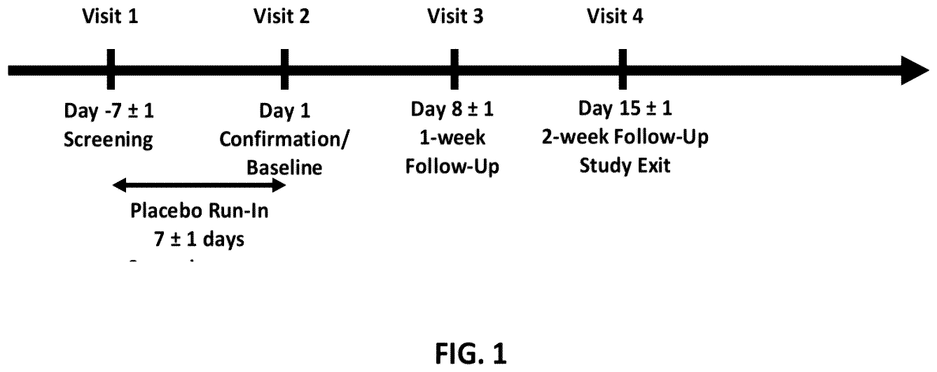

FIG. 1 shows a study schedule that included 4 weekly visits. During the first visit (Day-7±1), the subjects were screened, evaluated before CAE®, exposed to CAE®, evaluated after CAE®, and started with the placebo run-in period (7±1 days). During the 7-day study run-in period (for the purpose of subject selection) prior to randomization, all subjects received Placebo OS bilaterally, three times daily (TID). During the second visit, subjects were evaluated before CAE®, exposed to CAE®, and evaluated after CAE®. These evaluations formed the baseline measurements. The subjects were then randomized into two groups to receive either BRM421 ophthalmic solution (OS) or Placebo OS (Vehicle) bilaterally three times daily (TID) for 14 days from Visit 2 to Visit 4.

During these visits, subjects were evaluated for symptom efficacy. Specifically, visual analog scale (VAS) was measured by asking subjects to rate each ocular symptom due to ocular dryness. Symptoms assessed include burning/stinging, itching, foreign body sensation, blurred vision, eye dryness, photophobia, and pain.

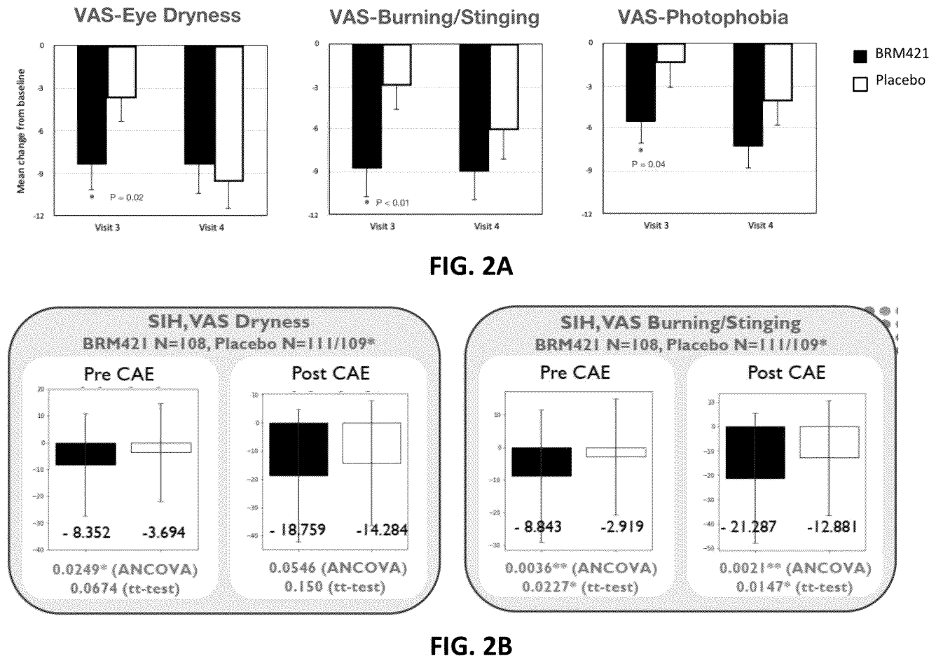

FIG. 2A shows results for VAS dryness, VAS burning/stinging, and VAS photophobia scales for the third and the fourth visits, relative to the second visit pre-CAE® evaluations as the baselines. As shown in FIG. 2A, both the BRM421 OS group and the placebo group showed improvements over the baseline. Compared to the placebo group, the BRM421 OS group showed a significant difference in mean changes from the baseline at Visit 3, but not at Visit 4, for improvements in burning/stinging, eye dryness, and photophobia in the intention-to-treat (ITT) Population. The lack of significant difference at VISIT 4 between the treatment group and the placebo group is because the vehicle also has some soothing effects that can alleviate/lessen symptoms over time. The fact that significant difference between the treatment group and the placebo group was observed at Visit 3 suggests that the effects of BRM421 is relatively fast onset.

FIG. 2B shows the results for VAS dryness and VAS burning/stinging scales for the third visit, relative to the second visit pre-CAE® evaluations or post-CAE® evaluations as the baselines. Using the pre-CAE® evaluations as the baselines, both the VAS dryness and the VAS burning/stinging showed significant improvements for the BRM421 group relative to the placebo group. Using the post-CAE® evaluations as the baselines, the VAS dryness showed less significant improvement for the BRM421 group relative to the placebo group, while the VAS burning/stinging showed significant improvement for the BRM421 group relative to the placebo group.

The results of VAS dryness, burning/stinging, and photophobia evaluations are summarized in TABLE 2.

TABLE 2
Visual Analog Scale (VAS) Data

| | | | |
|---|---|---|---|
| | | BRM421 OS | Placebo |
| Visit (Study Day) | Statistic | (N = 108) | (N = 112) |

Burning/Stinging

| Change from | n | 108 | 111 |
|---|---|---|---|
| Baseline at Visit 3 | Mean (SD) | −8.8 (20.35) | −2.9 (17.79) |
| (Day 8) | ANCOVA LS Mean^{b} (SE) | −9.8 (1.67) | −3.0 (1.65) |
| | ANCOVA LS Mean | −6.8 (2.31) | |
| | Difference^{a} (SE) | | |
| | ANCOVA Two-Sided 95% | (−11.4, −2.2) | |
| | CI^{b} | | |
| | ANCOVA p-value^{b} | 0.0036 | |
| | Two-Sample t-test Sample | −5.9 (2.58) | |
| | Mean Difference^{a} (SE) | | |
| | Two-Sample t-test Two- | (−11.0, −0.8) | |
| | Sided 95% CI | | |
| | Two-Sample t-test p-value | 0.0227 | |
| | Wilcoxon Rank Sum Test | 0.0884 | |
| | p-value | | |
| Change from | n | 108 | 111 |
| Baseline at Visit 4 | Mean (SD) | −9.0 (21.26) | −6.1 (20.92) |
| (Day 15) | ANCOVA LS Mean^{b} (SE) | −10.0 (1.86) | −6.2 (1.84) |
| | ANCOVA LS Mean | −3.8 (2.58) | |
| | Difference^{a} (SE) | | |
| | ANCOVA Two-Sided 95% | (−8.9, 1.3) | |
| | CI^{b} | | |
| | ANCOVA p-value^{b} | 0.1427 | |
| | Two-Sample t-test Sample | −2.9 (2.85) | |
| | Mean Difference^{a} (SE) | | |
| | Two-Sample t-test Two- | (−8.5, 2.7) | |
| | Sided 95% CI | | |
| | Two-Sample t-test p-value | 0.3113 | |
| | Wilcoxon Rank Sum Test | 0.3454 | |
| | p-value | | |

Eye Dryness

| Change from | n | 108 | 111 |
|---|---|---|---|
| Baseline at Visit 3 | Mean (SD) | −8.4 (19.22) | −3.7 (18.29) |
| (Day 8) | ANCOVA LS Mean^{b} (SE) | −9.4 (1.69) | −4.1 (1.66) |
| | ANCOVA LS Mean | −5.3 (2.33) | |
| | Difference^{a} (SE) | | |
| | ANCOVA Two-Sided 95% | (−9.9, −0.7) | |
| | CI^{b} | | |
| | ANCOVA p-value^{b} | 0.0249 | |
| | Two-Sample t-test Sample | −4.7 (2.53) | |
| | Mean Difference^{a} (SE) | | |
| | Two-Sample t-test Two- | (−9.7, 0.3) | |
| | Sided 95% CI | | |
| | Two-Sample t-test p-value | 0.0674 | |
| | Wilcoxon Rank Sum Test | 0.0269 | |
| | p-value | | |
| Change from | n | 108 | 111 |
| Baseline at Visit 4 | Mean (SD) | −8.4 (21.48) | −9.6 (19.97) |
| (Day 15) | ANCOVA LS Mean^{b} (SE) | −9.2 (1.92) | −10.0 (1.89) |
| | ANCOVA LS Mean | 0.8 (2.66) | |
| | Difference^{a} (SE) | | |
| | ANCOVA Two-Sided 95% | (−4.5, 6.0) | |
| | CI^{b} | | |
| | ANCOVA p-value^{b} | 0.7731 | |
| | Two-Sample t-test Sample | 1.2 (2.80) | |
| | Mean Difference^{a} (SE) | | |
| | Two-Sample t-test Two- | (−4.3, 6.7) | |
| | Sided 95% CI | | |
| | Two-Sample t-test p-value | 0.6700 | |
| | Wilcoxon Rank Sum Test | 0.6816 | |
| | p-value | | |

Photophobia

| Change from | n | 108 | 111 |
|---|---|---|---|
| Baseline at Visit 3 | Mean (SD) | −5.6 (15.49) | −1.4 (18.72) |
| (Day 8) | ANCOVA LS Mean^{b} (SE) | −6.2 (1.59) | −1.7 (1.56) |
| | ANCOVA LS Mean | −4.5 (2.19) | |
| | Difference^{a} (SE) | | |
| | ANCOVA Two-Sided 95% | (−8.8, −0.2) | |
| | CI^{b} | | |
| | ANCOVA p-value^{b} | 0.0415 | |
| | Two-Sample t-test Sample | −4.2 (2.33) | |
| | Mean Difference^{a} (SE) | | |
| | Two-Sample t-test Two- | (−8.8, 0.4) | |
| | Sided 95% CI | | |
| | Two-Sample t-test p-value | 0.0732 | |
| | Wilcoxon Rank Sum Test | 0.0626 | |
| | p-value | | |
| Change from | n | 108 | 111 |
| Baseline at Visit 4 | Mean (SD) | −7.3 (16.50) | −4.1 (18.88) |
| (Day 15) | ANCOVA LS Mean^{b} (SE) | −7.8 (1.63) | −4.3 (1.61) |
| | ANCOVA LS Mean | −3.5 (2.26) | |
| | Difference^{a} (SE) | | |
| | ANCOVA Two-Sided 95% | (−8.0, 0.9) | |
| | CI^{b} | | |
| | ANCOVA p-value^{b} | 0.1192 | |
| | Two-Sample t-test Sample | −3.2 (2.40) | |
| | Mean Difference^{a} (SE) | | |
| | Two-Sample t-test Two- | (−7.9, 1.5) | |
| | Sided 95% CI | | |
| | Two-Sample t-test p-value | 0.1838 | |
| | Wilcoxon Rank Sum Test | 0.2334 | |
| | p-value | | |

Abbreviations: ANCOVA = Analysis of Covariance; CAE ® = Controlled Adverse Environment; CI = Confidence Interval; LS = Least Squares; SE = Standard Error.
^{a}Mean Difference = BRM421 OS − Placebo
^{b}ANCOVA Model, ANCOVA p-value calculated using a model with treatment arm, site, and baseline value.
Note:
N in headers represents the total number of subjects enrolled in each respective treatment arm within the ITT population. Subject was asked to rate their eye dryness in each eye (OU) by placing a vertical mark on the horizontal line to indicate their current level of discomfort. 0 mm corresponds to “No Discomfort,” and 100 mm corresponds to “Maximal Discomfort.” The length of the assessment line is 100 mm.
Baseline measures are defined as the last Pre-CAE ® measure prior to the initiation of study treatment.

Subjects were also asked to grade the severity of their dry eye disease symptoms in their diary in the morning and in the evening before instilling the study drug. The Ora Ocular Discomfort & 4-Symptom Questionnaire (ODS Questionnaire) was used, which included rating the severity of 5 symptoms: ocular discomfort, burning, dryness, grittiness, and stinging. Each symptom rating ranged from 0 to 5, where 0=None and 5=Worst.

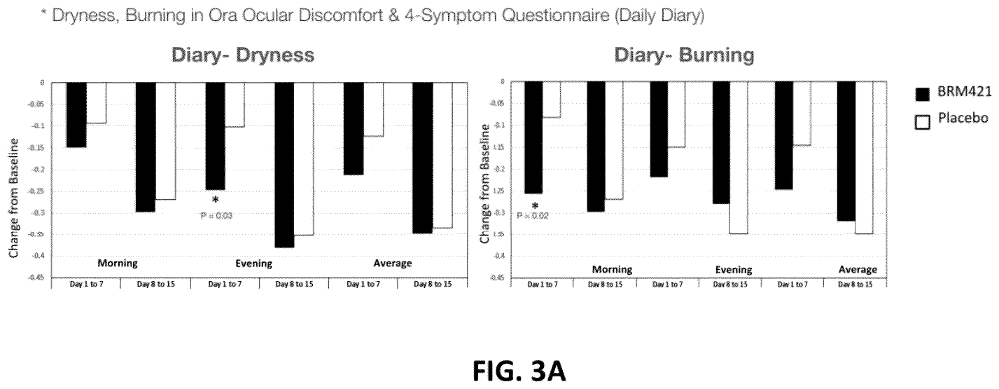

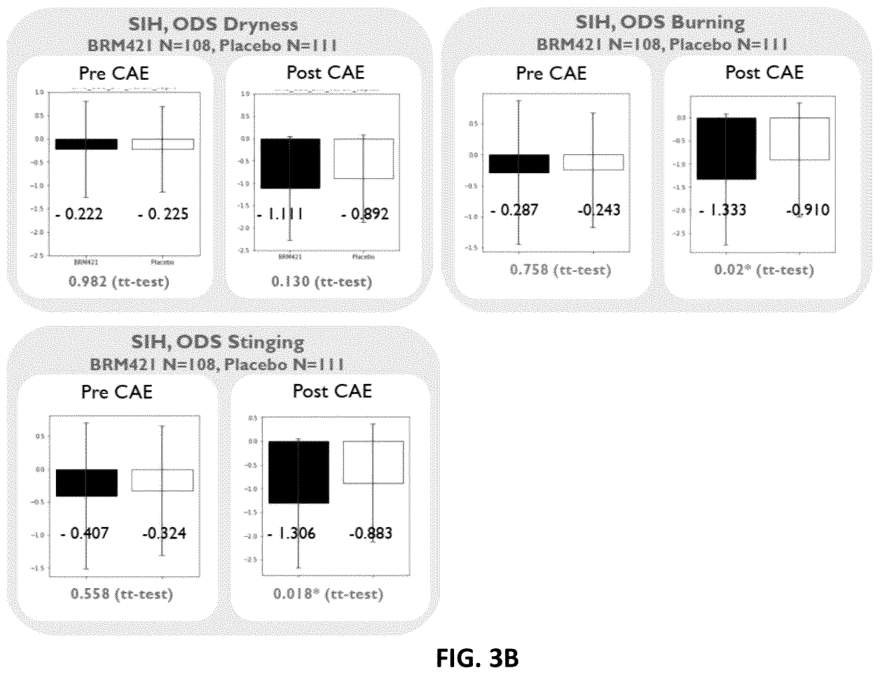

FIG. 3A shows daily diary results for dryness and burning. Compared to the placebo group, the BRM421 OS group showed a significantly better improvement in dryness from the baseline (pre-CAE®) from Day 1 to Day 7 (Visit 2 to Visit 3) in the evening, but not significantly better in the morning, nor from Day 8 to Day 15 (Visit 3 to Visit 4). The fact that the improvement was not significantly better in the morning is likely because the dryness was not serious in the morning after a long night sleep (shut eyes). The fact that no better improvement was observed for the BRM421 OS group, as compared to the placebo group, from Day 8 to Day 15 (Visit 3 to Visit 4) suggests that the effects of BRM421 is relatively fast onset, and the slow soothing effects of vehicle eventually caught up with the effects of BRM421.

With respect to ocular discomfort and burning, the BRM421 OS group showed significantly better improvements from the baseline (pre-CAE®) from Day 1 to Day 7 (Visit 2 to Visit 3) in the morning, as compared to the placebo group. However, compared to the placebo group, the better improvements of the BRM421 OS group were not significant in the evening, nor from Day 8 to Day 15 (Visit 3 to Visit 4). The fact that no better improvement was observed for the BRM421 OS group, as compared to the placebo group, from Day 8 to Day 15 (Visit 3 to Visit 4) suggests that the effects of BRM421 is relatively fast onset, and the slow soothing effects of vehicle eventually caught up with the effects of BRM421.

FIG. 3B shows the results for ODS dryness, ODS burning, and ODS stinging for the third visit, relative to the second visit pre-CAE® evaluations and post-CAE® evaluations as the baselines. For ODS dryness, there was no significant difference between the BRM421 group and the placebo group for both pre-CAE® post-CAE® evaluations as the baselines. For ODS burning and ODS stinging, the BRM421 group showed significant effects relative to the placebo group in the post-CAE® evaluations, but not in the pre-CAE® evaluations.

Results from the Ocular Discomfort & 4-Symptom Questionnaire (ODS) Diary data are summarized in TABLE 3.

TABLE 3
Ocular Discomfort & 4-Symptom Questionnaire (Diary) -
ITT Population with Observed Data Only

| | | | |
|---|---|---|---|
| | | BRM421 OS | Placebo |
| Visit (Study Day) | Statistic | (N = 108) | (N = 112) |

Ocular Discomfort

| Change from | n | 108 | 110 |
|---|---|---|---|
| Baseline to Day | Mean (SD) | −0.149 (0.5064) | 0.003 (0.5642) |
| 1 to 7, Morning | ANCOVA LS Mean^{b} (SE) | −0.138 (0.0518) | 0.008 (0.0512) |
| | ANCOVA LS Mean | −0.146 (0.0717) | |
| | Difference^{a} (SE) | | |
| | ANCOVA Two-Sided 95% | (−0.288, −0.005) | |
| | CI^{b} | | |
| | ANCOVA p-value^{b} | 0.0428 | |
| | Two-Sample t-test Sample | −0.152 (0.0727) | |
| | Mean Difference^{a} (SE) | | |
| | Two-Sample t-test Two- | (−0.295, −0.009) | |
| | Sided 95% CI | | |
| | Two-Sample t-test p-value | 0.0378 | |
| | Wilcoxon Rank Sum Test | 0.1023 | |
| | p-value | | |
| Change from | n | 108 | 111 |
| Baseline to Day | Mean (SD) | −0.248 (0.6696) | −0.190 (0.7143) |
| 8 to 15, Morning | ANCOVA LS Mean^{b} (SE) | −0.223 (0.0656) | −0.174 (0.0646) |
| | ANCOVA LS Mean | −0.049 (0.0906) | |
| | Difference^{a} (SE) | | |
| | ANCOVA Two-Sided 95% | (−0.227, 0.130) | |
| | CI^{b} | | |
| | ANCOVA p-value^{b} | 0.5913 | |
| | Two-Sample t-test Sample | −0.058 (0.0936) | |
| | Mean Difference^{a} (SE) | | |
| | Two-Sample t-test Two- | (−0.242, 0.127) | |
| | Sided 95% CI | | |
| | Two-Sample t-test p-value | 0.5391 | |
| | Wilcoxon Rank Sum Test | 0.6376 | |
| | p-value | | |

Burning

| Change from | n | 108 | 110 |
|---|---|---|---|
| Baseline to Day | Mean (SD) | −0.256 (0.5580) | −0.084 (0.5375) |
| 1 to 7, Morning | ANCOVA LS Mean^{b} (SE) | −0.254 (0.0528) | −0.081 (0.0522) |
| | ANCOVA LS Mean | −0.173 (0.0731) | |
| | Difference^{a} (SE) | | |
| | ANCOVA Two-Sided 95% | (−0.317, −0.029) | |
| | CI^{b} | | |
| | ANCOVA p-value^{b} | 0.0186 | |
| | Two-Sample t-test Sample | −0.172 (0.0742) | |
| | Mean Difference^{a} (SE) | | |
| | Two-Sample t-test Two- | (−0.318, −0.026) | |
| | Sided 95% CI | | |
| | Two-Sample t-test p-value | 0.0214 | |
| | Wilcoxon Rank Sum Test | 0.0695 | |
| | p-value | | |
| Change from | n | 108 | 111 |
| Baseline to Day | Mean (SD) | −0.332 (0.7199) | −0.284 (0.6691) |
| 8 to 15, Morning | ANCOVA LS Mean^{b} (SE) | −0.313 (0.0640) | −0.265 (0.0630) |
| | ANCOVA LS Mean | −0.048 (0.0884) | |
| | Difference^{a} (SE) | | |
| | ANCOVA Two-Sided 95% | (−0.222, 0.126) | |
| | CI^{b} | | |
| | ANCOVA p-value^{b} | 0.5871 | |
| | Two-Sample t-test Sample | −0.048 (0.0939) | |
| | Mean Difference^{a} (SE) | | |
| | Two-Sample t-test Two- | (−0.233, 0.137) | |
| | Sided 95% CI | | |
| | Two-Sample t-test p-value | 0.6082 | |
| | Wilcoxon Rank Sum Test | 0.3944 | |
| | p-value | | |

Dryness

| Change from | n | 108 | 110 |
|---|---|---|---|
| Baseline to Day | Mean (SD) | −0.241 (0.5137) | −0.102 (0.4802) |
| 1 to 7, Evening | ANCOVA LS Mean^{b} (SE) | −0.243 (0.0484) | −0.098 (0.0479) |
| | ANCOVA LS Mean | −0.145 (0.0671) | |
| | Difference^{a} (SE) | | |
| | ANCOVA Two-Sided 95% | (−0.277, −0.012) | |
| | CI^{b} | | |
| | ANCOVA p-value^{b} | 0.0321 | |
| | Two-Sample t-test Mean | −0.140 (0.0673) | |
| | Difference^{a} (SE) | | |
| | Two-Sample t-test Two- | (−0.272, −0.007) | |
| | Sided 95% CI | | |
| | Two-Sample t-test p-value | 0.0392 | |
| | Wilcoxon Rank Sum Test | 0.0528 | |
| | p-value | | |
| Change from | n | 107 | 110 |
| Baseline to Day | Mean (SD) | −0.382 (0.6917) | −0.353 (0.6554) |
| 8 to 15, Evening | ANCOVA LS Mean^{b} (SE) | −0.377 (0.0651) | −0.342 (0.0642) |
| | ANCOVA LS Mean | −0.036 (0.0901) | |
| | Difference^{a} (SE) | | |
| | ANCOVA Two-Sided 95% | (−0.213, 0.142) | |
| | CI^{b} | | |
| | ANCOVA p-value^{b} | 0.6937 | |
| | Two-Sample t-test Sample | −0.029 (0.0915) | |
| | Mean Difference^{a} (SE) | | |
| | Two-Sample t-test Two- | (−0.209, 0.151) | |
| | Sided 95% CI | | |
| | Two-Sample t-test p-value | 0.7531 | |
| | Wilcoxon Rank Sum Test | 0.8508 | |
| | p-value | | |

Abbreviations: ANCOVA = Analysis of Covariance; CI = Confidence Interval; LS = Least Squares; SE = Standard Error.
^{a}Mean Difference = BRM421 OS − Placebo
^{b}ANCOVA Model, ANCOVA p-value calculated using a model with treatment arm, site, and baseline value.
Note:
N in headers represents the total number of subjects enrolled in each respective treatment group within the ITT population. Ora Calibra ® Ocular Discomfort & 4-Symptom Questionnaire ranged from 0 to 5.
A score of 0 = None and 5 = Worst.

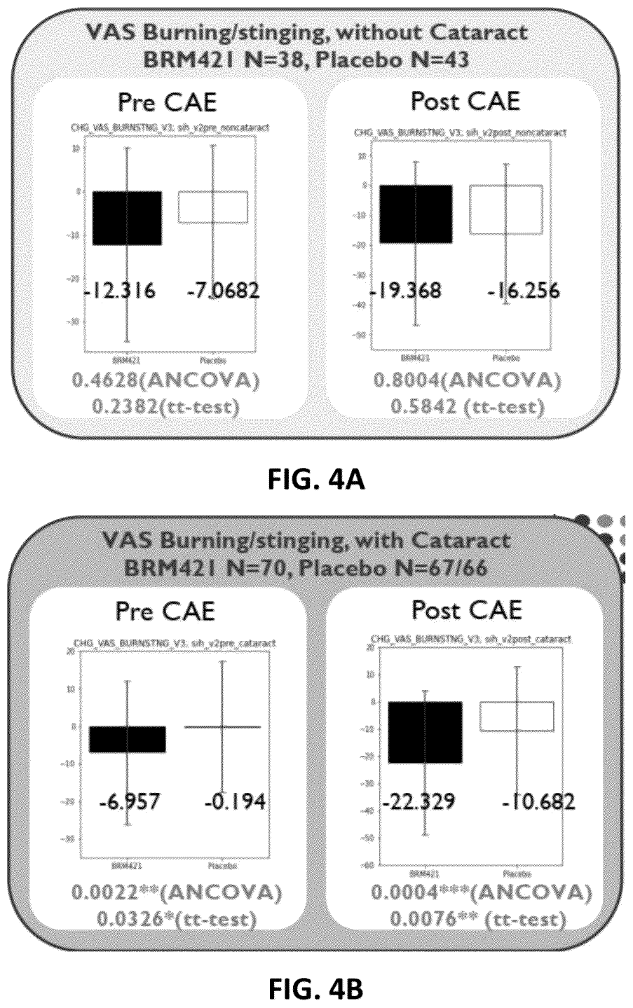

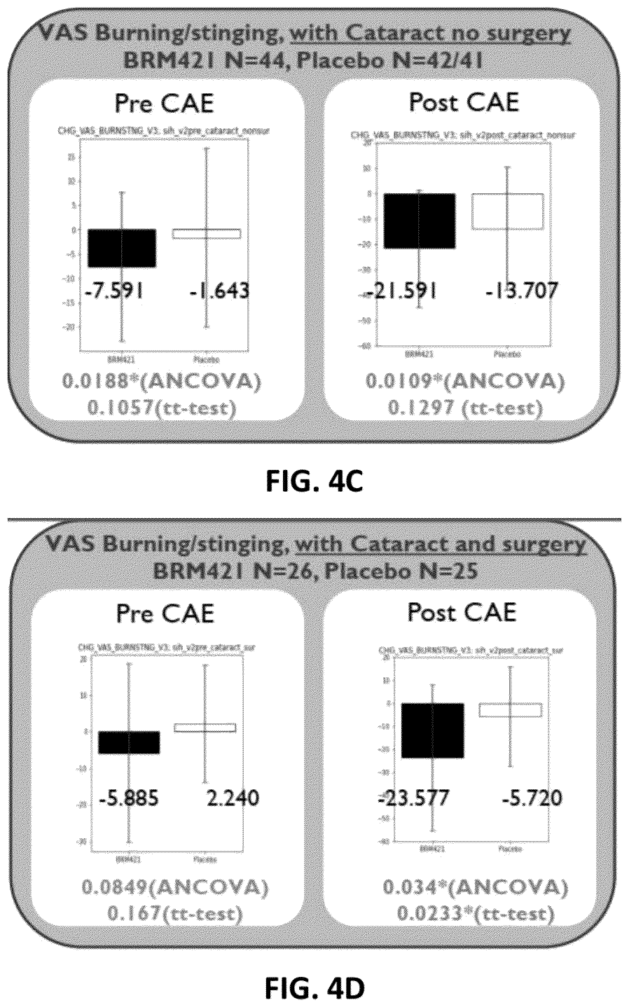

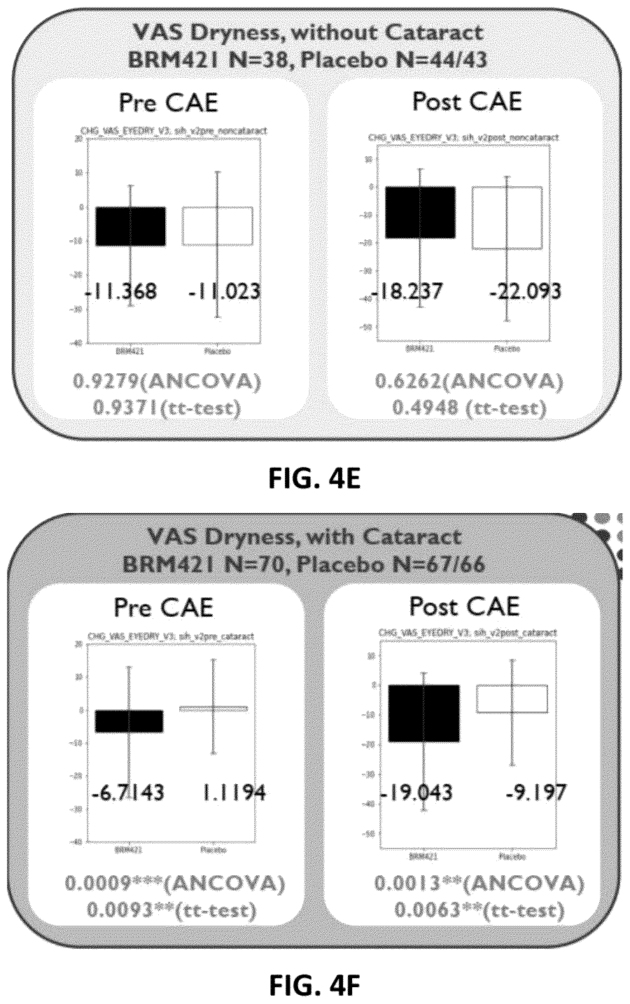

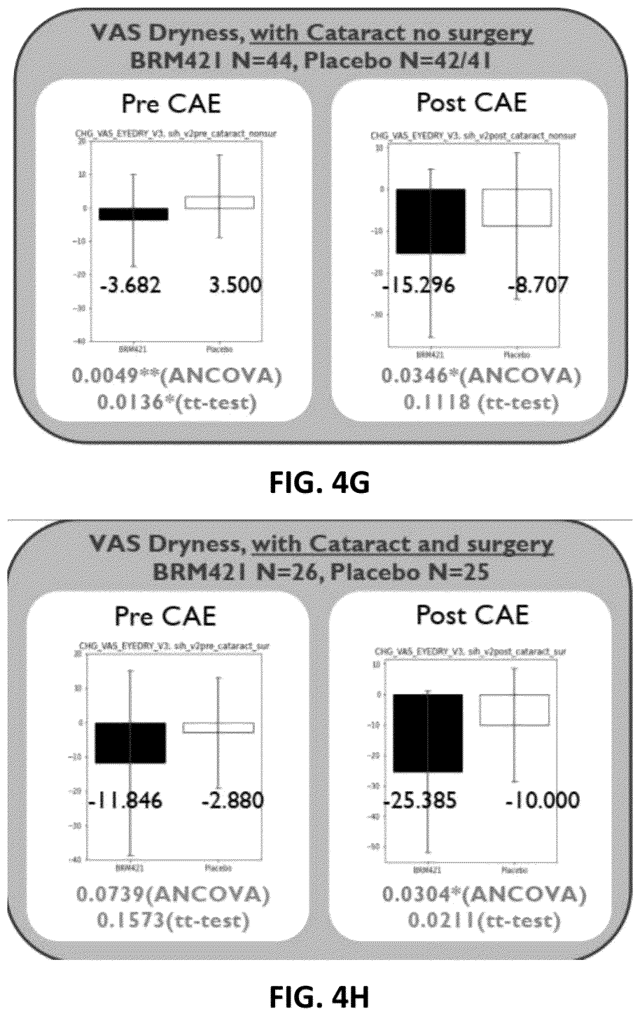

Cataract is a cloudy area in the lens of eyes. The prevalence of both dry eye and cataract increases with age. Thus, it is not uncommon for patients to suffer from DED and cataract at the same time. Subjects in the above BRM421 group and the placebo group are further analyzed in sub-groups based on whether they also suffer from cataract. This analysis unexpectedly revealed that the BRM421 treatments showed no significant improvements in VAS burning/stinging in patients with only DED, based on either pre-CAE® or post-CAE® evaluations (see FIG. 4A). However, the BRM421 treatments showed very significant improvements in VAS burning/stinging in patients with both DED and cataract, based on both pre-CAE® and post-CAE® evaluations (see FIG. 4B).

Cataract is typically treated with surgery. DED may complicate cataract surgery or post-op effects. In addition, cataract surgery may exacerbate pre-existing dry eye diseases or induce dry eye in patients with healthy corneas. The postoperative dry eye may impact visual outcomes and visual recovery time. Thus, the subjects with both DED and cataract were further analyzed based on whether they had cataract surgery. As shown in FIG. 4C and FIG. 4D, the BRM421 treatments showed significant improvements in VAS burning/stinging with or without cataract surgery, as compared to the placebo group. Nevertheless, the BRM421 treatments showed more improvements in VAS burning/stinging in subjects with cataract surgery (see FIG. 4D), as compared to subjects without cataract surgery (see FIG. 4C). These results suggests that the BRM421 treatment would be particularly effective in treating DED in patients with cataract surgery.

When analyzed with respect to VAS dryness, the same results were found. As shown in FIG. 4E, the BRM421 treatments did not show significant improvements (based on both pre-CAE® and post-CAE® evaluations) in subjects without cataract, as compared to the placebo group. On the other hand, the BRM421 treatments showed significant improvements (based on both pre-CAE® and post-CAE® evaluations) in subjects with cataract, as compared to the placebo group (see FIG. 4F). In addition, the BRM421 treatments showed significant improvements (based on both pre-CAE® and post-CAE® evaluations) in subjects with cataract regardless of the surgery status (see FIG. 4G and FIG. 4H).

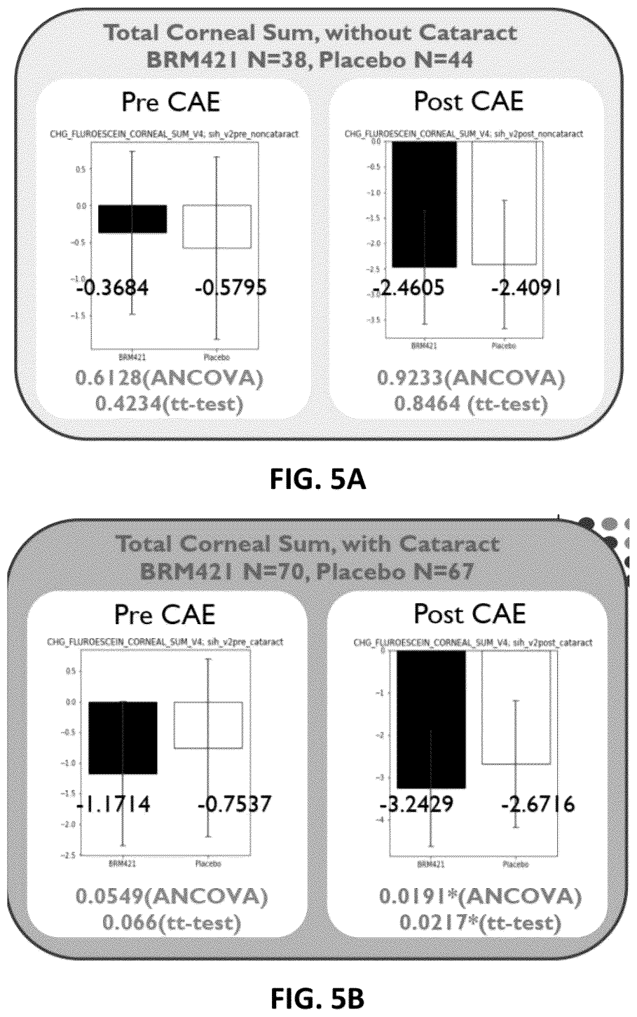

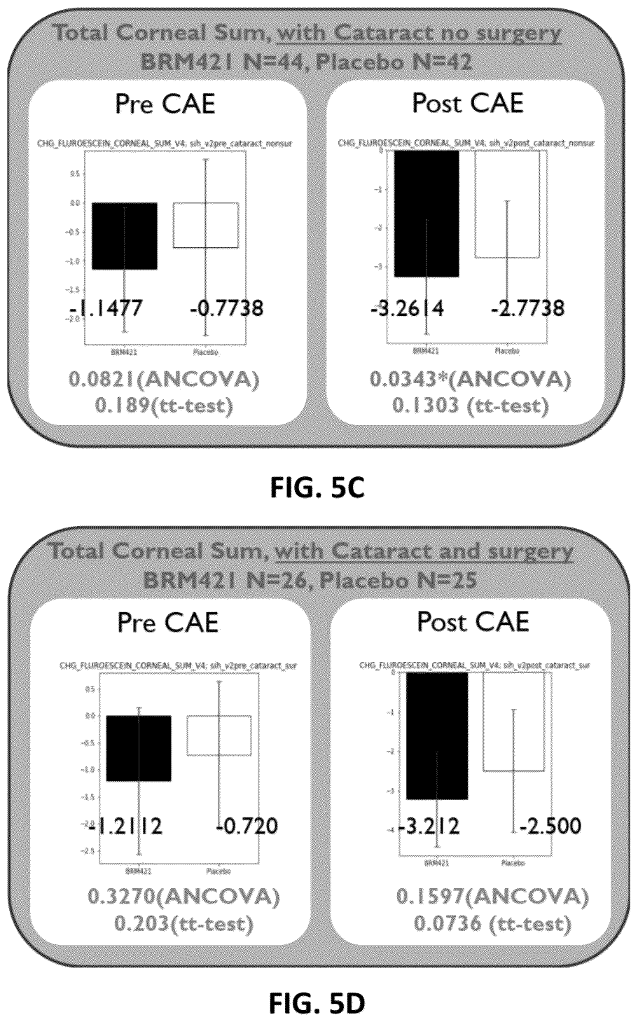

Severe dry eye cases can lead to cornea damages, which can be assessed with fluorescein staining. PDSP of the invention can treat DED. In addition, these PDSP can also repair corneal damages. As shown in FIG. 5A, BRM421 treatments did not show significant improvements in corneal damage repair in subjects without cataract. In contrast, FIG. 5B shows that BRM421 treatments demonstrated significant improvements in corneal damage repair in subjects with cataract. These improvements seem more significant with subjects having cataract without surgery (FIG. 5C), as compared to subjects having cataract with surgery (FIG. 5D). The selective efficacies of BRM421 treatment in cataract patients are unexpected.

The above results clearly demonstrate the efficacies of PDSP of the invention in the treatment of DED. In particular, these PDSP were unexpected found to be very effective in treating or preventing DED in subjects with cataract, before or after surgery. Embodiments of the invention relate to pharmaceutical compositions and methods for preventing and/or treating dry eye in a subject. A subject in accordance with embodiments of the invention may be a human or an animal. A method in accordance with an embodiment of the invention may comprise administering to a subject in need of dry eye prevention or treatment with a pharmaceutical composition comprising a peptide selected from any PDSP listed in TABLE 1. In accordance with examples of the invention, the pharmaceutical composition may comprise a peptide of the invention, or a salt of such a peptide, together with a pharmaceutically acceptable carrier or excipient, such as distill water, saline, oil, or gel.

A pharmaceutical composition of the invention may be formulated in any suitable dosage forms, such as a solution, an ointment, a suspension, a gel, or an emulsion, which may be formulated at any suitable concentrations, such as 10-200 μM. One skilled in the art would be able to formulate these at a suitable concentration to deliver an effective dose without inventive efforts. These dosage forms may be formulated for topical application to the eyes or other suitable routes of administrations (e.g., oral or injection).

While embodiments of the invention have been illustrated with a limited number of examples. One skilled in the art would appreciate that other modifications or variations are possible without departing from the scope of the invention. Therefore, the scope of protection should be limited by the accompanying claims.

1. (canceled)

2. (canceled)

3. (canceled)

4. A method for treating dry eye disease, comprising: administering to a subject in need thereof a composition comprising a peptide having the amino-acid sequence selected from SEQ ID NO: 1-7.

5. The method according to claim 4, wherein the peptide having the amino-acid sequence of SEQ ID NO: 3.

6. The method according to claim 4, wherein the subject suffers from cataract.

7. The method according to claim 4, wherein the subject had cataract that has been treated with surgery.

8. The method according to claim 5, wherein the subject suffers from cataract.

9. The method according to claim 5, wherein the subject had cataract that has been treated with surgery.


SEQUENCE LISTING (7 sequences)

SEQ ID NO: 1 - protein, 39 aa, Homo sapiens
LSVATALSALSLGAEQRTESIIHRALYYDLISSPDIHGT

SEQ ID NO: 2 - protein, 34 aa, Homo sapiens
ALSALSLGAEQRTESIIHRALYYDLISSPDIHGT

SEQ ID NO: 3 - protein, 29 aa, Homo sapiens
SLGAEQRTESIIHRALYYDLISSPDIHGT

SEQ ID NO: 4 - protein, 24 aa, Homo sapiens
SLGAEQRTESIIHRALYYDLISSP

SEQ ID NO: 5 - protein, 20 aa, Homo sapiens
SLGAEQRTESIIHRALYYDL

SEQ ID NO: 6 - protein, 29 aa, Mus musculus
SLGAEHRTESVIHRALYYDLITNPDIHST

SEQ ID NO: 7 - protein, 20 aa, Mus musculus
SLGAEHRTESVIHRALYYDL